(19) United States
(12) Patent Application Publication
(10) Pub. No.: US 2024/0024140 A1
(43) Pub. Date: Jan. 25, 2024

(54) Balloon Tipped Dual Lumen Tapered Guide Extension Catheter

(71) Applicant: Hady LICHAA, Murfreesboro, TN (US)

(72) Inventor: Hady LICHAA, Murfreesboro, TN (US)

(21) Appl. No.: 18/480,753

(22) Filed: Oct. 4, 2023

Related U.S. Application Data
(63) Continuation-in-part of application No. 18/121,399, filed on Mar. 14, 2023.
(60) Provisional application No. 63/200,135, filed on Feb. 16, 2021.

(51) Int. Cl. A61F 2/958; A61F 2/966

(52) U.S. Cl. CPC A61F 2/958; A61F 2/966

(57) ABSTRACT

The present disclosure discloses a guide extension catheter device. The catheter device includes an elongated body having at least one of a distal portion, a proximal portion, and a middle portion. The middle portion extends between the distal portion and the proximal portion. In an aspect, the distal portion comprises a first part and a second part. The first part is disposed at a distal end of the distal portion and the second part is disposed at a proximal end of the distal portion. The first part of the distal portion is cylindrical in shape and the second part is tapered such that a diameter of a second part proximal end is greater than a diameter of a second part distal end. The catheter device also integrates a built-in balloon at the first part of the distal end.

### CROSS-REFERENCE TO RELATED APPLICATIONS

This Application is a Continuation-in-Part of U.S. application Ser. No. 18/121,399, filed Mar. 14, 2023, to Lichaa, titled “Balloon Tipped Dual Lumen Tapered Guide Extension Catheter,” which claims priority to U.S. Provisional Patent Application No. 63/320,135, filed Mar. 15, 2022, titled, “Balloon Tipped Dual Lumen Tapered Guide Extension Catheter,” the contents of each are incorporated by reference in their entireties.

### FIELD OF THE DISCLOSURE

The present invention relates to a catheter, and more specifically, to a tapered end of a catheter and a built-in balloon at its tip with a separate accessible lumen.

### BACKGROUND

Guide extension catheters play an essential part in complex non-surgical interventions in the heart arteries. For example, the guide extension catheters create a smooth pathway for balloon and/or stent delivery. Typically, the guide extension catheters use a sheath to guide or ease insertion of surgical instruments, such as balloons, into the heart arteries.

Conventional catheters are susceptible to causing tear or injury while being delivered in the heart arteries. In addition, it may be difficult to deliver large equipment in the heart arteries using the conventional catheters. Furthermore, vessel injury or dissection is known to occur when balloon is delivered into the heart arteries using the conventional catheters. In a traditional catheter a mid-section of the catheter is made up of a supple half-pipe which is a u-shaped high-sided ramp or a runway.

U.S. Patent Publication No. US20170028170A1 to Andrew Ho (2016) provides a guide catheter extension device which helps in assisting the delivery of interventional cardiology devices to a treatment site within a patient.

In the referred reference, a guide catheter extension device is made up of a flexible, elongate extension catheter having a tapered tip portion at a distal end, an opening at a proximal end, and a body portion extending between the two. The extension catheter has a longitudinal slit extending from the distal tip portion toward the proximal opening. Methods of using the guide catheter extension device to aid in performing interventional cardiology procedures.

U.S. Pat. No. 10,946,177B2 to Dean Peterson (2019) relates to a guide extension catheter to be used with guide catheters.

In the referred reference, a guide extension catheter for use with a predefined length guide catheter and related methods for treating blood vessel lesions and abnormalities is disclosed. A guide extension catheter may include a push member, an elongate tube member, and a balloon radially surrounding a portion of the elongate tube member. The balloon may include an inflatable tube coupled to an elongate shaft (shown as shaft 302 in FIG. 3) having a lumen for receiving inflation fluid, and the inflatable tube can be coiled in a helical manner around the elongate tube member. A bio-active layer may coat an outer surface portion of the balloon and, when the balloon is inflated, one or more drugs of the bio-active layer can be received by the blood vessel. The inflation of the balloon may engage the elongate tube member with an inner surface of the blood vessel and/or an inner surface of the guide catheter.

Thus, there is a need for a guide extension catheter that can effectively and easily deliver the equipment in the heart arteries, without causing injury or tear.

### BRIEF SUMMARY OF THE PRESENT INVENTION

The present disclosure discloses a guide extension catheter. The guide extension catheter may include an elongated body having a distal portion, a proximal portion, and a middle portion. The middle portion may extend between the distal portion and the proximal portion. In some aspects, the distal portion may include a first part and a second part. The first part may be disposed at a distal end of the distal portion, and the second part may be disposed at a proximal end of the distal portion. In some aspects, the first part may be cylindrical in shape having a constant diameter, and the second part may be conical in shape. In particular, the second part may be tapered such that a diameter of a second part proximal end may be greater than a diameter of a second part distal end. In further aspects, the first part may have a length that may be shorter than a length of the second part.

The guide extension catheter may further include a built-in balloon that may be integrated at a first part distal end. The guide extension catheter may further include a sliding tab configured to control sliding movement of the built-in balloon. In particular, the sliding tab may advance and retract the built-in balloon within and outside the first part distal end. In further aspects, the sliding tab may lock position of the built-in-balloon. The sliding tab may further control movement of a built-in-balloon shaft. In some aspects, the sliding tab may be disposed at the proximal portion of the guide extension catheter.

The guide extension catheter may additionally include a first radio-opaque marker and a second radio-opaque marker. The first radio-opaque marker may be disposed at the first part distal end, and the second radio-opaque marker may be disposed at an intersection point of the first part and the second part of the distal portion. In some aspects, the first radio-opaque marker and the second radio-opaque marker may be circular in shape.

In further aspects, the guide extension catheter may include a central rod that may be disposed at the proximal portion. The central rod may include two channels for “Over-the-wire” (OTW) and “Rapid-exchange” (Rx) lumens. In additional aspects, the guide extension catheter may include a diagonally sliced multi-layered pipe at the middle portion.

In some aspects, the guide extension catheter includes an elongated body having a distal portion and a proximal portion. The distal portion includes a first part and a second part. The first part is disposed at a distal end of the distal portion, and the second part is disposed at a proximal end of the distal portion. The first part is cylindrical in shape and the second part is tapered such that a diameter of a second part proximal end is greater than a diameter of a second part distal end. The guide extension catheter further includes a shaft configured to slide in the elongated body. The shaft is disposed at a lower portion of the elongated body. The guide extension catheter further includes a built-in balloon integrated on a distal end of the shaft. The built-in balloon is configured to slide within the first part and outside the first part towards a first part distal end of the elongated body. The built-in balloon is configured to inflate from the lower portion to an upper portion of the elongated body to prevent movement of an equipment (such as a wire) over the built-in balloon.

In some aspects, the built-in balloon covers an entire circumference of the elongated body when the built-in balloon is inflated in the first part. In further aspects, the distal end of the shaft is prevented to slide inside the second part.

In some aspects, the shaft includes a first radio-opaque marker and a second radio-opaque marker disposed at a shaft distal end, and the built-in balloon is disposed over the first radio-opaque marker and the second radio-opaque marker. The guide extension catheter further includes a third radio-opaque marker disposed at the first part distal end, and a fourth radio-opaque marker disposed at an intersection point of the first part and the second part.

In further aspects, the guide extension catheter includes a sliding tab configured to control sliding movement of the shaft. The sliding tab is disposed at the proximal portion. The sliding tab is further configured to lock position of the shaft.

The present disclosure is further directed to a method that includes providing a built-in balloon inside a distal portion of a guide extension catheter device. The built-in balloon is integrated on a distal end of a shaft disposed at a lower portion of an elongated body of the guide extension catheter device. The elongated body includes a distal portion and a proximal portion. The distal portion includes a first part and a second part. The first part is disposed at a distal end of the distal portion, and the second part is disposed at a proximal end of the distal portion. The first part is cylindrical in shape and the second part is tapered such that a diameter of a second part proximal end is greater than a diameter of a second part distal end. The built-in balloon is configured to slide within the first part and outside the first part towards a first part distal end of the elongated body, and the built-in balloon is configured to inflate from the lower portion to an upper portion of the elongated body to prevent movement of an equipment (such as a wire) over the built-in balloon. The method further includes inflating the built-in balloon inside the first part to cover an entire circumference of the elongated body to prevent movement of the equipment over the built-in balloon.

The guide extension catheter provides various advantages over conventional guide extension catheters. The guide extension catheter minimizes vessel injury, for example, due to tapered shape of the distal portion of the guide extension catheter. In addition, tapering the tip of the guide extension catheter facilitates deeper and safer insertion of the guide catheter extension, thereby delivering equipment (e.g., large equipment) to heart arteries more effectively. Further, the guide extension catheter allows an operator to insert the equipment without a sheath placement. Furthermore, built-in balloon eliminates the need of an external balloon in the guide extension catheter. In addition, the built-in balloon is configured to prevent movement of an additional equipment (such as a wire) over the balloon which saves procedural time and radiation (e.g., when the built-in balloon is inflated). Stated another way, the built-in balloon traps the additional equipment in the distal portion of the guide extension catheter, and does not allow the additional equipment to move over the built-in balloon until the built-in balloon is deflated.

### BRIEF DESCRIPTION OF THE DRAWINGS

FIG. 1 is a perspective view of guide extension catheter device, according to one embodiment.

FIG. 2 is a perspective view of a guide extension catheter device with a diagonally sliced multi-layered pipe, according to one embodiment.

FIG. 3 illustrates a perspective view of another guide extension catheter device, according to one embodiment.

FIGS. 4A, 4B, 4C, and 4D illustrate catheter delivery techniques associated with the other guide extension catheter device, according to one embodiment.

### DETAILED DESCRIPTION OF EMBODIMENTS

The invention now will be described more fully hereinafter with reference to the accompanying drawings, in which embodiments of the invention are shown. This invention may however be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present therebetween. As used herein, the term “and/or” includes any and all combinations of one or more of the associated listed items.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section.

It will be understood that the elements, components, regions, layers and sections depicted in the figures are not necessarily drawn to scale.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom,” “upper” or “top,” “left” or “right,” “above” or “below,” “front” or “rear,” may be used herein to describe one element's relationship to another element as illustrated in the Figures. It will be understood that relative terms are intended to encompass different orientations of the device in addition to the orientation depicted in the Figures.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. The numbers, ratios, percentages, and other values may include those that are ±5%, ±10%, ±25%, ±50%, ±75%, ±100%, ±200%, ±500%, or other ranges that do not detract from the spirit of the invention. The terms about, approximately, or substantially may include values known to those having ordinary skill in the art. If not known in the art, these terms may be considered to be in the range of up to ±5%, ±10%, or other value higher than these ranges commonly accepted by those having ordinary skill in the art for the variable disclosed. Thus, embodiments of the present invention should not be construed as limited to the particular shapes of regions illustrated herein but are to include deviations in shapes that result, for example, from manufacturing. The invention illustratively disclosed herein suitably may be practiced in the absence of any elements that are not specifically disclosed herein. All patents, patent applications and non-patent literature cited through this application are hereby incorporated by reference in their entireties.

The addition of a balloon at the tip of these catheters minimizes vessel injury. The presence of a balloon that could be retracted into the catheter also allows for “trapping” of wires which simplifies and facilitates equipment exchanges in and out of the heart. This also allows the delivery of catheters from very small wrist arteries to the heart with the least risk for vessel injury. it also allows the operator to choose the smallest equipment profile to get to the heart through very small wrist arteries. Moreover, the tapering of the catheter tip allows for deeper and safer insertion of the catheter extension into the heart arteries which allows operators to fix heart arteries much easier and faster. Finally, the presence of two lumens through the catheter extension opens up new opportunities for the physician to be more creative in crossing complex heart blockages.

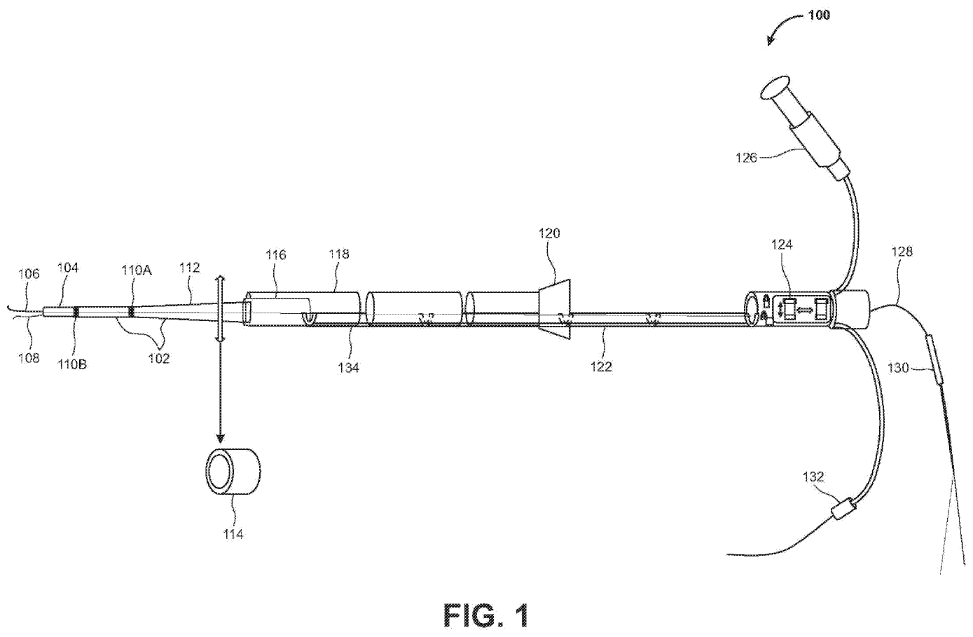

A guide extension catheter device 100 with a supple half-pipe 134 is disclosed in FIG. 1, according to one embodiment. The device includes a guide extension catheter 102 that combines many functions of multiple useful pieces of equipment into one. It does not only serve the goal of delivering equipment to the heart arteries more effectively but also safely, while offering a cost advantage by combining many tools into one.

It has a built-in balloon at its tip which is slidable in and out of the catheter. The balloon can also be semi-compliant without detracting from the spirit of the invention. The balloon can be fully retracted within the distal end of the catheter. The balloon employed here may be compliant balloon 104 which are elastorrteric in nature and are typically made of polyurethane or silicone. Generally, the compliant balloon 104 is inflated by a measure of volume instead of pressure. The compliant balloon 104 may be inflated within the range between 100% -800%. This compliant balloon 104 has a lumen accessible through a separate port at the proximal end of the catheter. The tip of the catheter is tapered which allows for better deliverability, tracking, pushability and less vessel injury.

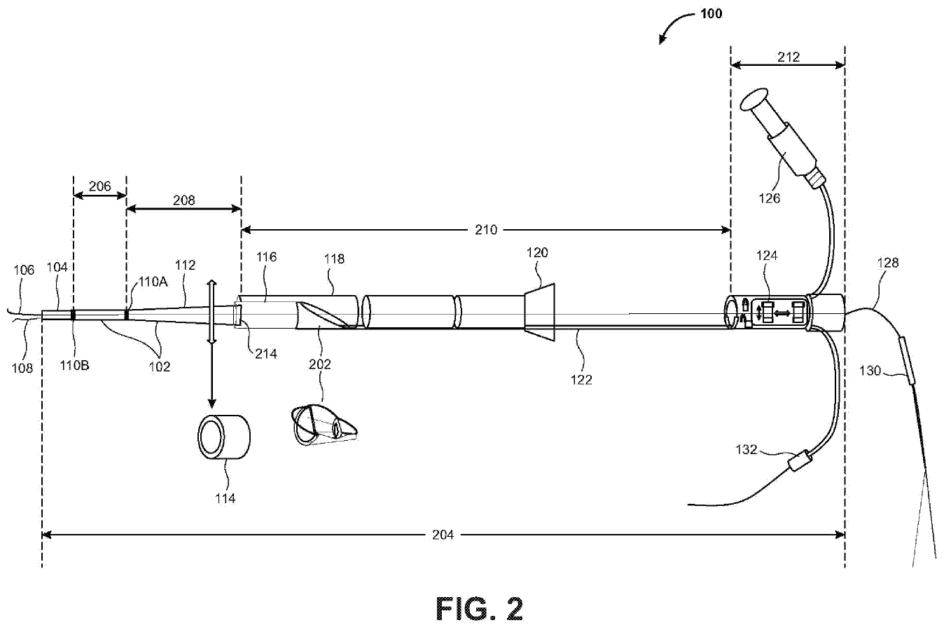

A guide extension catheter device 100 with a diagonally sliced multi-layered pipe 202 is disclosed in FIG. 2, according to another embodiment. The mid-section of the catheter is made up of a diagonally sliced multi-layered pipe 202, with a top sliced edge shaped as an ellipse in the diagonal axis, which enhances catheter movement. The bottom side of the sliced pipe is connected firmly to the central rod 122. The external layer of the sliced pipe has a polymer jacket 112 which enhances catheter lubricity and movement within the guide catheter. The middle-braided layer 114 of the sliced pipe is continuous with the middle-braided layer 114 of cylindrical distal segment of the catheter. This segment provides more effective forward force transmission, which physically translates into better catheter pushability.

The range of motion of the balloon 104 along the longitudinal axis of the distal guide extension catheter 118 can be controlled with a sliding tab 124 that is part of the proximal part of the catheter. Wherein, the balloon 104 is a part of the catheter.

The proximal end of the catheter transitions to a full cylinder which is mounted by a movable tab that controls the sliding of the distal balloon at the tip of the catheter. There is also a locking function (here locking tab)which locks the balloon in the same position. The most proximal end of the catheter trifurcates into three separate parts: The rnain central port, the “Over the wire” balloon port and the built-in catheter balloon in-deflator 126.

Description and functionality of this Catheter: The catheter has two separate lumens. The first lumen is the largest one and is the continuation of the proper inner lumen of the guide extension catheter 102. This lumen is labeled “Rapid Exchange (Rx) port” 106 since it is the lumen of the “Rapid exchange” guide extension tube. The second lumen is a small channel in the shaft of the sliding balloon at the tip of the catheter. This second lumen is connected to the proximal “over-the-wire” port through a small tube that runs through the shaft of the guide extension, its central rod, the shaft of the proximal cylindrical end of the catheter and finally through a dedicated extension tubing connected to the proximal portion 212 of the catheter. The extension tubing ends with a wire hub on its proximal end which helps with threading of an wire.

The compliant or semi-compliant balloon 104 at the tip of the catheter is short and is connected through a small tube that runs through the distal guide extension catheter 118 and central rod to a tube extending separately from the proximal end of the catheter. This tube is also hubbed with a Luer-lock adaptor which allows the connection to a built-in catheter balloon in-deflator 126. The balloon shaft can be slid in both forward and backward directions in a rail which is carved on the inner surface of the guide extension catheter 102.

The sliding balloon shaft is connected to a stiff rod which runs inside a channel constructed between the layers of the half-cylindrical mid portion of the catheter. In one of its embodiments, this stiff rod is connected to a sliding tab 124 on the external surface of the cylindrical proximal end of the catheter. This sliding tab 124 can be advanced in both forward and backwards relative to the longitudinal axis of the catheter. The tab can be moved in a fixed locked position and in a direction perpendicular to the longitudinal axis of the catheter. This locking mechanism allows the operator to slide the distal balloon back and forth relative to the tip of the guide extension then lock it into the same position. The operator can then unlock the balloon position and move it to a different spot relative to the tip of the extension catheter. The longitudinal range of motion of the balloon may extend from completely outside the catheter extension (ahead of its tip) to the most distal portion 110A of the conical segment of the guide extension catheter 102.

The distal portion of the guide extension catheter 102 is made up of a cylindrical portion most distally, connected proximally to a partial cone shaped tube, which has a gradually larger inner diameter from its distal to its proximal sub-segment. Hence the catheter extension is tapered in the proximal to distal direction until it reaches a prespecified inner diameter, then the inner diameter is constant through its most distal short sub-segment. There is a circular radio-opaque marker (here first radio-opaque marker 110A) built into the catheter at the transition between to the conical proximal sub-segment and the cylindrical distal sub-segment. There is also another built-in circular radio-opaque marker (here second radio-opaque marker 110B) at the distal tip of the catheter.

The distal tip is cylindrical in shape with a fixed diameter along a short distance. The tapering starts in the mid portion of the guide extension to its proximal segment where it meets the push rod. The configuration of cylindrical distal portion and the conical proximal portion 212 is unique. In one of its embodiments, the proximal portion 212 has a movable tab which slides back and forth allowing the distal balloon to advance and retract within and outside the most distal end of the catheter. Furthermore, we have different tapering options for the most proximal segment of the guide extension.

In one of its embodiments, the longer half pipe extends throughout the majority of the push rod length. The push rod centers the half-pipe. The half pipe configuration allows for more effective transmission of forward force to the distal guide extension catheter 118 and allows for better pushability through complex coronary anatomy.

The guide extension catheter 102 is multi-layered and braided, as shown in a cross-sectional view in FIG. 1 and FIG. 2. The central layer is a made up of multiple braided metallic wires. The outer layers are made up of a synthetic fluoro-polymer. Its inner surface is covered with a hydrophilic layer. Its outer surface is covered with a polymer jacket 112 and a hydrophilic component. The half pipe is reinforced by a layer of braided wires and the half-pipe connecting to the proximal rod is much larger allowing more force transmission from the rod to the half-pipe.

The mid portion of the catheter may be a semi-cylindrical shape centered by a stiff flat rod. The lateral wings connected to the central rod 122 are made of a flexible plastic material which allows it to be compressed and collapsed at the proximal opening of the hemostatic valve 120 connected to the guide catheter. The closure of the hemostatic valve 120 should allow for a good seal after the introduction of the guide extension catheter 102 into the guide catheter lumen. The central rod 122 has 2 built-in channels for the “Over-the-wire” (OTW) and “Rapid-exchange” Rx lumens as described above.

The proximal port 132 at the proximal end of the catheter may be cylindrical in shape and connected to the proximal end of the central rod 122. The outer surface harbors the sliding tab 124 which controls the position of the distal balloon as described above. The tab can be locked in any position along its rail, through the perpendicular displacement of the button into the locking position. This proximal segment of the catheter is connected to two extension tubings. One of them is the OTW distal port 108 and the other is the Rapid Exchange (Rx) port 106 connected to the balloon lumen. Wherein, the workhorse guidewire 128 is inserted in the main vessel either through a Rapid Exchange (Rx) port 106 or an over-the-wire (OTW) distal port 108. A bio-active layer may coat an outer surface portion of the balloon and, when the balloon is inflated, one or more drugs of the bio-active layer can be received by the blood vessel. The bio-active layer used here to coat the balloon can be a chemical coating known as polytetrafluoroethylene (PTFE) coating 116.

Additionally, a locking function which locks a position of the distal balloon in the same position as relative to the guide extension catheter 102. This function does not use a spiraling balloon around the distal end of the catheter but a different mechanism that fixes the balloon in the same position.

In other aspects, the integrated built-in balloon shaft slides along a closed rail system may be locked in a certain position along its range of motion. The locking mechanism may include a “push-pull” swiveling tab which can be fixated in an “On” or “Off” position, depending on the direction of its tilting axis, towards or away from the distal tip of the catheter respectively. Another locking mechanism may include a hemostatic valve which could be tightened around the shaft of the sliding balloon. Clockwise rotation of the hemostatic wheel tightens the grip on the sliding balloon shaft and counter-clockwise rotation of the hemostatic wheel loosens the grip on the sliding balloon. The range of motion of the balloon may include a portion or the entire length of the guide extension as well as a portion of the entire length of the proximal guide extension shaft. The range of motion of the proximal end of the built-in balloon may extend distally beyond the distal tip of the guide extension.

This catheter is drastically different than other guide extension catheters 102 on the current market. Not only it may significantly improve the performance of current guide extension catheters but it also adds four other functionalities, which currently require four other pieces of equipment. Firstly, the advantage of this catheter is that it is tapered which significantly improves its deliverability through turns/tortuosity, its tracking and pushability through calcified arteries.

Secondly, tapering of the catheter allows the operator to deliver a guiding catheter through a vessel access site without the use of a sheath, in other words, in a sheathless fashion, hence saving another dedicated piece of equipment and decreasing the size of hole in the access vessel, which translates into less bleeding and vascular complications. This reduces the morbidity of a procedure without affecting the functionality of a guide extension catheter 102. It also allows the operator to use large pieces of equipment through the radial artery (in the wrist) which otherwise could only be delivered through the femoral artery (in the groin), hence minimizing the procedural risk while increasing patient safety, satisfaction, and hospital length of stay (healthcare cost advantage). Moreover, usual procedures that could not be performed from the radial artery (in the wrist) due to the very small size of this vessel in some patients, can now be performed in a sheathless fashion, with the help of the tapered piece of equipment.

Thirdly, the tapered tip and the built-in balloon (also unique to this catheter) acting as a dilator, minimizes vessel injury/dissection during delivery. There is an additional price advantage by saving the cost of an additional balloon. The built-in balloon also allows for the application of the three or three delivery techniques without additional equipment: Balloon assisted tracking, balloon surfing and inch-warming techniques.

Fourthly, another advantage of having the built-in balloon at the tip of the catheter instead of proximal to the guide extension portion (like it exists currently in the “Trapliner” catheter made by Teleflex) is that it allows trapping wires much closer to the interventional area of interest, in the same fluoroscopic field, which saves procedural time and radiation, since this maneuver is repeated multiple times during complex interventions.

Fifthly, the “built-in” portion of the balloon through the shaft of the catheter saves precious space in the inner lumen of the catheter which could be used for additional equipment to be delivered simultaneously, hence decreasing the size of the required guiding catheter, thereby decreasing the size of the hole through the accessed artery which translates clinically to less bleeding and vascular complications.

Finally, this tool adds another function to this catheter which currently requires an additional expensive piece of equipment—A dual lumen catheter. This allows the use of this catheter in complex anatomy where the operator needs to get to a separate difficult space/branch without losing access to the main vessel. The dual lumen function also allows the operator to maintain wire access while injecting drugs/contrast distally or while aspirating an intra-mural hematoma in chronic total occlusion related work. The lumen goes through the balloon shaft to the tip of the balloon to allow the passage of an additional wire.

This catheter can be made in multiple diameters which could fit in a 6, 7 or 8 French guiding catheters (size of the outer diameter of the largest portion of the distal end of the catheter extension) and with multiple tip sizes ranging from 5.5 to 7 French. It can also be made in different lengths ranging from 120 to 150 cm. The length of the distal segment of the catheter (connected to the semi-cylindrical mid portion) can also vary in lengths from 10 to 25 cm.

This catheter is designed to be used in the coronary arteries, cerebral arteries and peripheral endovascular space.

The embodiments provide for several advantages over the prior art. For example:

1. Tapered tip allows better deliverability, safer transition in heart arteries and allows deeper intubation of the heart arteries which facilitates access to difficult areas and the delivery of a stent 130 through tortuous vessel segments.

2. Built-in sliding balloon at the tip which allows for catheter delivery through the balloon surfing, balloon assisted tracking and inch-warming techniques without the use of an additional and separate coronary balloon. The details of the techniques are described below.

3. Allows the operator to insert equipment in very small wrist arteries without a sheath placement.

4. The position of the balloon at the tip allows for trapping and exchanging gear much easier and faster than the existing “Trapliner” (made by Teleflex).

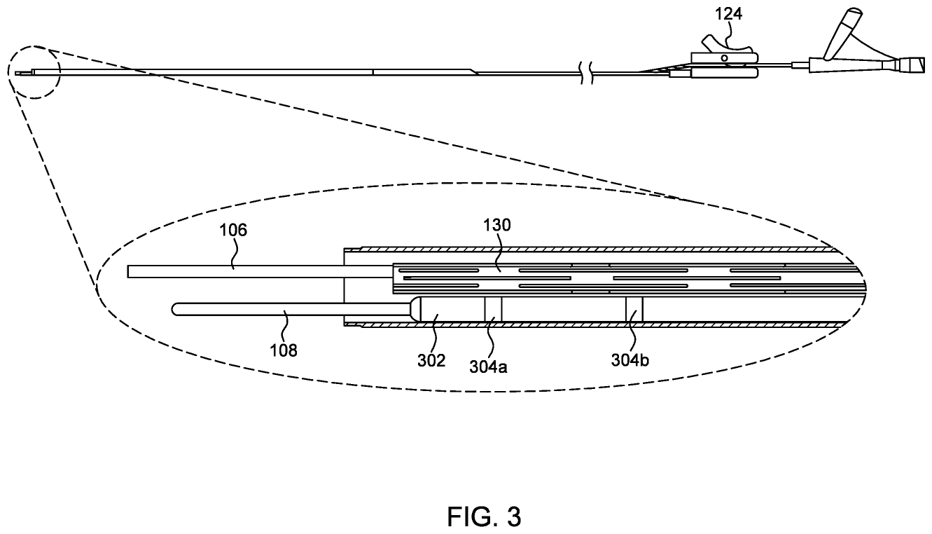

In accordance with the present disclosure, the guide extension catheter 102 includes the distal end 206, 208 and the proximal end 212. The distal end 206, 208 includes an integrated balloon 104 and the proximal end 212 includes a sliding tab 124 (e.g., a swiveling locking tab configured to move in a lock/unlock position) that controls movement of a built-in-balloon shaft 302 (e.g., relative to the tip of the guide extension catheter 102) disposed at the distal end of the guide extension catheter 102 (as shown in FIG. 3). The shaft 302 is disposed at a lower portion of the guide extension catheter 102. In some aspects, the balloon 104 is integrated on the shaft 302 (e.g., at a distal end of the shaft 302), and the balloon 104 moves as the shaft 302 moves. The shaft includes a third radio-opaque marker 304A and a fourth radio-opaque marker 304B. The third radio-opaque marker 304A and the fourth radio-opaque marker 304B are disposed at a shaft distal end, and at a predefined distance from each other. The balloon 104 is disposed over the third radio-opaque marker 304A and the fourth radio-opaque marker 304B. The third radio-opaque marker 304A and the fourth radio-opaque marker 304B facilitate in determining the position of the balloon 104.

As described above, the guide extension catheter 102 includes the first part distal portion 206 and the second part distal portion 208. The first part distal portion 206 is cylindrical in shape and the second part distal portion 208 is tapered such that a diameter of a second part proximal end 214 is greater than a diameter of a second part distal end. Further, the balloon 104 is integrated at a first part distal end.

In some aspects, the shaft 302 is configured to move the balloon 104 back and forth in the first part distal portion 206. The balloon 104 is trapped in the first part distal portion 206 such that the balloon 104 cannot be pulled in other portions/length of the guide extension catheter 102 (such as the second part distal portion 208). The shaft 302 is configured to move the balloon 104 outside the first part distal portion 206 from the distal end of the guide extension catheter 102 such that the balloon 104 guides the movement of the guide extension catheter 102.

In some aspects, the location of the balloon 104 relative to the first part distal portion 206 is determined using the first radio-opaque marker 110A, the second radio-opaque marker 110B, the third radio-opaque marker 304A, and the fourth radio-opaque marker 304B.

The built-in balloon 104 is inflated in an eccentric manner (e.g., from an original position to an inflated position). For example, the balloon 104 inflates from the shaft 302 (located in proximity to the lower portion or a bottom end/wall of the guide extension catheter 102) and moves towards an upper portion or upper end/wall of the guide extension catheter 102, in some aspects, the balloon 104 is inflated such that the balloon 104 touches the upper wall. Stated another way, the balloon 104 is inflated from bottom to top of the guide extension catheter 102 and blocks movement of the wire 106 (or any other equipment) over the balloon 104. In addition, the balloon 104 is configured to be deflated from the inflated position to the original position (or any other position) such that the stent 130 is moved above the deflated balloon 104, within the lumen of the guide extension, at the time of delivery of the stent 130 to the coronary lesion to be treated. Stated another way, the balloon 104 is deflated such that a space is created for the stent 130 to pass over the deflated balloon 104. In some aspects, the balloon 104 is deflated from the inflated position to the original position (or any other position).

As described above, the guide extension catheter 102 includes double lumen. The double lumen includes a first lumen and a second lumen. The first lumen is “Rapid Exchange (Rx) port” 106 and the second lumen is a small channel in the shaft 302 of the sliding balloon 104 at the tip of the catheter. The second lumen is connected to the proximal “over-the-wire” port through a small tube that runs through the shaft of the guide extension, its central “half-pipe” shaped rod, the shaft of the proximal cylindrical end of the catheter and finally through a dedicated extension tubing connected to the proximal portion 212 of the catheter.

In some aspects, the “Rapid Exchange (Rx) port” 106 is configured to facilitate delivery of an equipment such as the stent 130. The guide extension catheter 102 is configured to enclose both the shaft 302 (with integrated balloon 104) and the stent 130. The stent 130 moves over the shaft 302 at the time of delivery of the stent 130 to its destination in the coronary artery.

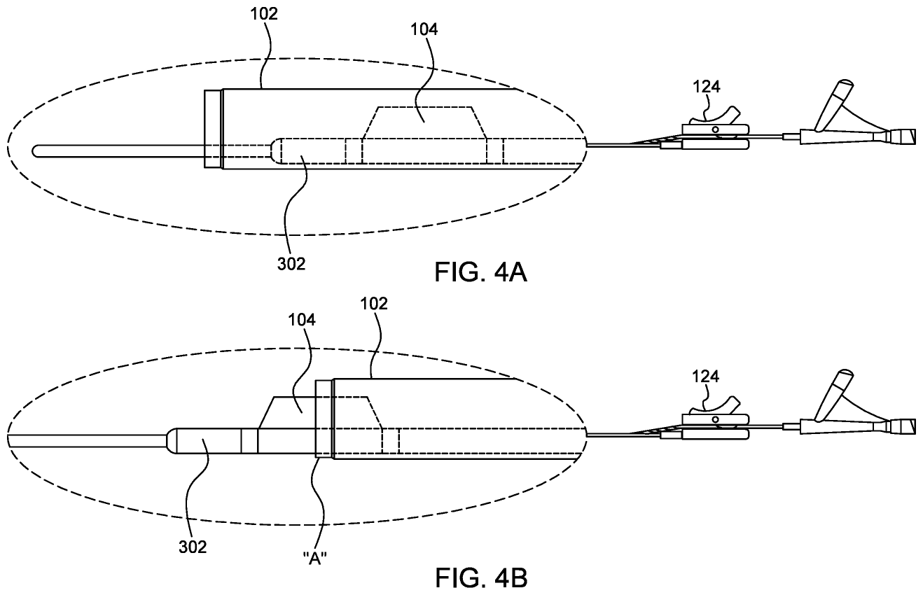

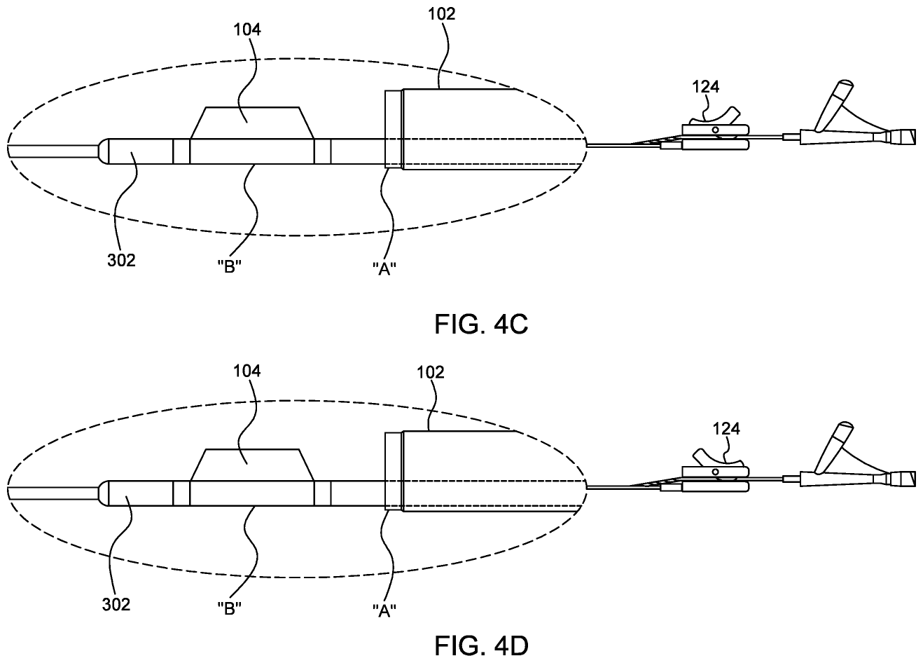

The catheter delivery techniques are explained in conjunction with FIGS. 4A-4D as follows:

Trapping: In trapping technique (as shown in FIG. 4A), the built-in balloon 104 is positioned inside first part distal portion 206, and inflated in the eccentric manner (e.g., from an original position to an inflated position) as described above. In some aspects, the balloon 104 is inflated such that the balloon 104 touches the upper wall/end of the guide extension catheter 102. In some aspects, the balloon 104 is inflated in the first part distal portion 206 and trapped in the first part distal portion 206. For example, the balloon 104 is inflated in a center portion of the first part distal portion 206. In some aspects, the balloon 104 is inflated in any position between edges of the first part distal portion 206 such that the balloon 104 is completely inside the first part distal portion 206. When the balloon 104 is inflated in the first part distal portion 206, the balloon 104 prevents movement of the equipment (such as the stent 130) over the balloon 104, as the balloon 104 is inflated. Stated another way, the balloon 104 is inflated such that the balloon 104 covers the entire circumference of the guide extension catheter 102. Further, the entire circumference corresponds to a balloon length. Thus, the balloon 104 traps the stent 130 (or other equipment) in the first part distal portion 206, when the balloon 104 is inflated (and the stent 130 is prevented to move over the balloon 104). Stated another way, the inflated balloon blocks movement of the stent 130 over the balloon 104. The trapping technique saves procedural time and radiation as the operator is not required to perform fluoroscopy to detect position of the stent 130. The inflated balloon traps the stent 130 in the first part distal portion 206 and thus provides indication to the operator when the stent 130 reaches the first part distal portion 206.

Balloon Surfing: In the balloon surfing technique (as shown in FIG. 4B), the built-in balloon 104 is positioned such that a first portion of the balloon 104 is inside the guide extension catheter 102 and a second portion of the balloon 104 is outside the guide extension catheter 102 (e.g., from the distal end of the guide extension catheter 102). In some aspects, the built-in balloon 104 position is locked relative to the guide extension catheter 102 (e.g., tip of the guide extensioncatheter 102) using the sliding tab 124 such that the first portion of the balloon 104 is inside the guide extension catheter 102 and the second portion of the balloon 104 is outside the guide extension catheter 102. For example, the operator may move the sliding tab 124 in an unlocked position, and advance the built-in balloon 104 until the first portion of the balloon 104 is inside the guide extension catheter 102 and the second portion of the balloon 104 is outside the guide extension catheter 102. When the first portion is inside and the second portion is outside, the operator may move the sliding tab 124 in a locked position to lock the movement of the built-in balloon 104 relative to the guide extension catheter 102. The balloon 104 is then inflated, and whole bundle is advanced to prevent any injury to the vessel during movement. In some aspen s, the first portion and the second portion are equal. In other aspen s, the first portion and the second portion are unequal. The ratio of the first portion and the second portion is customized by the operator. In some aspects, the operator uses the first radio-opaque marker 110A, the second radio-opaque marker 110B, the third radio-opaque marker 304A, and the fourth radio-opaque marker 304B to determine the length of the first portion and the second portion, and customize the position of the balloon 104 based on the determination.

Balloon Assisted Tracking: In the balloon assisted tracking (as shown in FIG. 4C), the balloon 104 assists movement of the guide extension catheter 102. In some aspects, the operator controls the sliding tab 124 (e.g., move the sliding tab 124 in the unlocked position) to move/advance the balloon 104 from a first position “A” to a second position “B” away from (or outside of) the guide extension catheter 102 (as depicted in FIG. 4C). When the balloon 104 reaches the position “B” (that is at a specific distance from the first position “A”), the balloon 104 is inflated and then the guide extension catheter 102 moves over the shaft 302 and towards the balloon 104 Thereafter, the balloon 104 is deflated and advanced to move from the position “B” further away from the tip of the guide extension catheter 102, and inflated again. Thereafter, the guide extension catheter 102 again moves over the shaft 302 and towards the balloon 104. Stated another way, the guide extension catheter 102 and the balloon 104 move such that the guide extension catheter 102 follows the balloon 104. This prevents any injury during equipment movement (e.g., guide extension movement) through the complex coronary anatomy. In this technique, the balloon 104 acts as anchor over which the equipment such as the stent 130 moves. In some aspects, the operator uses the first radio-opaque marker 110A, the second radio-opaque marker 110B, the third radio-opaque marker 304A, and the fourth radio-opaque marker 304B to perform the balloon assisted tracking technique.

Inch-warming techniques: In the inch-warming technique (as shown in FIG. 4D), once the balloon 104 reaches the position “B” (as determined using the first radio-opaque marker 110A, the second radio-opaque marker 110B, the third radio-opaque marker 304A, and the fourth radio-opaque marker 304B), the operator deflates the balloon 104 (e.g., using the deflator 126). When the balloon 104 is deflating/deflated, the guide extension catheter 102 is moved/advanced over the balloon 104.

Clause 1. A guide extension catheter device 100 comprising: an elongated body 204 having a distal portion (206 & 208), a proximal portion 212, and a middle portion 210 extending between the distal portion and the proximal portion 212, wherein: the distal portion comprises a first part distal portion 206 and a second part distal portion 208, the first part is disposed at a distal end of the distal portion, the second part is disposed at a proximal end of the distal portion, the first part is cylindrical in shape and the second part is tapered such that a diameter of a second part proximal end 214 is greater than a diameter of a second part proximal end (herein 110A); and a built-in balloon integrated at a first part distal end.

The guide extension catheter device 100 has an elongated body 204 starting from a first part distal portion 206 displayed at an extreme left end to a second part proximal end 214 displayed at an extreme right, as displayed in the FIG. 2. The first part distal portion 206 has a cylindrical shape with a constant diameter. The second part distal portion 208 is conical in shape. The second part distal portion 208 has a second part distal end (herein with reference to first radio-opaque marker 110A) and a second part proximal end 214. The second part distal portion 208 is tapered such that a diameter of second part proximal end 214 is greater than a diameter of a second part proximal end (herein with reference to second radio-opaque marker 110A). Furthermore, the length of the first part distal portion 206 is shorter than the length of the second part distal portion 208.

A proximal portion 212 is located an extreme right end of the guide extension catheter device 100, wherein the sliding tab 124 is disposed at the proximal portion 212. A middle portion 210 is extended between the distal portion (here first part distal portion 206- and second-part distal portion 208) and the proximal portion 212.

Clause 2. The guide extension catheter device 100 of clause 1 further comprising: a first radio-opaque marker 110A disposed at the first part distal end; and a second radio-opaque marker 110B disposed at an intersection point of the first part and the second part.

Clause 3. The guide extension catheter device 100 of clause 2, wherein the first radio-opaque marker and the second radio-opaque marker are circular in shape.

Clause 4. The guide extension catheter device 100 of clause 1 further comprises a sliding tab 124 configured to control sliding movement of the built-in balloon.

Clause 5. The guide extension catheter device 100 of clause 4, wherein the sliding tab 124 is disposed at the proximal portion 212.

Clause 6. The guide extension catheter device 100 of clause 4, wherein the sliding tab 124 is further configured to advance and retract the built-in balloon within and outside the first part distal end.

Clause 7. The guide extension catheter device 100 of clause 4, wherein the sliding tab 124 is further configured to lock position of the built-in balloon.

Clause 8. The guide extension catheter device 100 of clause 4 further comprises a shaft associated with the built-in balloon, wherein the sliding tab 124 is configured to move the shaft to slide the built-in balloon.

Clause 9. The guide extension catheter device 100 of clause 1, wherein the middle portion 210 comprises a diagonally sliced multi-layered pipe 202.

Clause 10. The guide extension catheter device 100 of clause 1, wherein the middle portion 210 further comprises a central rod 122.

Clause 11. The guide extension catheter device 100 of clause 10, wherein the central rod 122 comprises two channels for “Over-the-wire” (OTW) and “Rapid-exchange” (Rx) lumens.

Clause 12. A guide extension catheter device 100 comprising: an elongated body 204 having a distal portion, a proximal portion 212, and a middle portion 210 extending between the distal portion and the proximal portion 212, wherein: the distal portion comprises a first part and a second part, the first part is disposed at a distal end of the distal portion, the second part is disposed at a proximal end of the distal portion, the first part is cylindrical in shape and the second part is tapered such that a diameter of a second part proximal end 214 is greater than a diameter of a second part distal end; a built-in balloon integrated at a first part distal end; and a central rod 122 disposed at the middle portion 210.

Clause 13. The guide extension catheter device 100 of clause 12 further comprising: a first radio-opaque marker 110A disposed at a first part distal end; and a second radio-opaque marker 110B disposed at intersection point of the first part and the second part.

Clause 14. The guide extension catheter device 100 of clause 13, wherein the first radio-opaque marker 110A and the second radio-opaque marker 110B are circular in shape.

Clause 15. The guide extension catheter device 100 of clause 12 further comprises a sliding tab 124 configured to control sliding movement of the built-in balloon.

Clause 16. The guide extension catheter device 100 of clause 15, wherein the sliding tab 124 is disposed at the proximal portion 212.

Clause 17. The guide extension catheter device 100 of clause 15, wherein the sliding tab 124 is further configured to advance and retract the built-in balloon within and outside the first part distal end.

Clause 18. The guide extension catheter device 100 of clause 15, wherein the sliding tab 124 is further configured to lock position of the built-in balloon.

Clause 19. The guide extension catheter device 100 of clause 12, wherein the middle portion 210 comprises a diagonally sliced multi-layered pipe 202.

Clause 20. A method for manufacturing a guide extension catheter device 100, the method comprising: providing an elongated body 204 having a distal portion, a proximal portion 212, and a middle portion 210 extending between the distal portion and the proximal portion 212, wherein: the distal portion comprises a first part distal portion 206 and a second part distal portion 208, the first part distal portion 206 is disposed at a distal end of the distal portion, the second part distal portion 208 is disposed at a proximal end of the distal portion, the first part is cylindrical in shape and the second part is tapered such that a diameter of a second part proximal end 214 is greater than a diameter of a second part distal end; integrating a built-in balloon at a first part distal end (herein 110A); and disposing a central rod 122 at the middle portion 210.

While the invention has been described in terms of exemplary embodiments, it is to be understood that the words that have been used are words of description and not of limitation. As is understood by persons of ordinary skill in the art, a variety of modifications can be made without departing from the scope of the invention defined by the following claims, which should be given their fullest, fair scope.

What is claimed is:

1. A guide extension catheter device comprising:
an elongated body having a distal portion and a proximal portion, wherein:
the distal portion comprises a first part and a second part,
the first part is disposed at a distal end of the distal portion,
the second part is disposed at a proximal end of the distal portion,
the first part is cylindrical in shape and the second part is tapered such that a diameter of a second part proximal end is greater than a diameter of a second part distal end; and
a shaft configured to slide in the elongated body, wherein the shaft is disposed at a lower portion of the elongated body; and
a built-in balloon integrated on a distal end of the shaft, wherein the built-in balloon is configured to slide within the first part and outside the first part towards a first part distal end of the elongated body, and wherein the built-in balloon is configured to inflate from the lower portion to an upper portion of the elongated body to prevent movement of an equipment over the built-in balloon.

2. The guide extension catheter device of claim 1, wherein the built-in balloon covers an entire circumference of the elongated body when the built-in balloon is inflated in the first part.

3. The guide extension catheter device of claim 1, wherein the distal end of the shaft is prevented to slide inside the second part.

4. The guide extension catheter device of claim 1, wherein the equipment is a stent.

5. The guide extension catheter device of claim 1, wherein the shaft comprises a first radio-opaque marker and a second radio-opaque marker disposed at a shaft distal end.

6. The guide extension catheter device of claim 5, wherein the built-in balloon is disposed over the first radio-opaque marker and the second radio-opaque marker.

7. The guide extension catheter device of claim 1 further comprising:
a third radio-opaque marker disposed at the first part distal end; and
a fourth radio-opaque marker disposed at an intersection point of the first part and the second part.

8. The guide extension catheter device of claim 1 further comprises a sliding tab configured to control sliding movement of the shaft.

9. The guide extension catheter device of claim 8, wherein the sliding tab is disposed at the proximal portion.

10. The guide extension catheter device of claim 8, wherein the sliding tab is further configured to lock position of the shaft.

11. A guide extension catheter device comprising:
an elongated body having a distal portion and a proximal portion, wherein:
the distal portion comprises a first part and a second part,
the first part is disposed at a distal end of the distal portion,
the second part is disposed at a proximal end of the distal portion,
the first part is cylindrical in shape and the second part is tapered such that a diameter of a second part proximal end is greater than a diameter of a second part distal end; and
a shaft configured to slide in the elongated body, wherein the shaft is disposed at a lower portion of the elongated body, wherein the shaft comprises a first radio-opaque marker and a second radio-opaque marker disposed at a shaft distal end; and
a built-in balloon integrated on a distal end of the shaft, wherein the built-in balloon is configured to slide within the first part and outside the first part towards a first part distal end of the elongated body, and wherein the built-in balloon is configured to inflate from the lower portion to an upper portion of the elongated body to prevent movement of an equipment over the built-in balloon.

12. The guide extension catheter device of claim 11, wherein the built-in balloon covers an entire circumference of the elongated body when the built-in balloon is inflated in the first part.

13. The guide extension catheter device of claim 11, wherein the distal end of the shaft is prevented to slide inside the second part.

14. The guide extension catheter device of claim 11, wherein the equipment is a stent.

15. The guide extension catheter device of claim 11, wherein the built-in balloon is disposed over the first radio-opaque marker and the second radio-opaque marker.

16. The guide extension catheter device of claim 11 further comprising:
a third radio-opaque marker disposed at the first part distal end; and
a fourth radio-opaque marker disposed at an intersection point of the first part and the second part.

17. The guide extension catheter device of claim 11 further comprises a sliding tab configured to control sliding movement of the shaft.

18. The guide extension catheter device of claim 17, wherein the sliding tab is disposed at the proximal portion.

19. The guide extension catheter device of claim 17, wherein the sliding tab is further configured to lock position of the shaft.

20. A method comprising:
providing a built-in balloon inside a distal portion of a guide extension catheter device, wherein the built-in balloon is integrated on a distal end of a shaft disposed at a lower portion of an elongated body of the guide extension catheter device, wherein:
the elongated body comprises a distal portion and a proximal portion,
the distal portion comprises a first part and a second part,
the first part is disposed at a distal end of the distal portion,
the second part is disposed at a proximal end of the distal portion,
the first part is cylindrical in shape and the second part is tapered such that a diameter of a second part proximal end is greater than a diameter of a second part distal end,
the built-in balloon is configured to slide within the first part and outside the first part towards a first part distal end of the elongated body,
the built-in balloon is configured to inflate from the lower portion to an upper portion of the elongated body to prevent movement of an equipment over the built-in balloon; and
inflating the built-in balloon inside the first part to cover an entire circumference of the elongated body to prevent movement of the equipment over the built-in balloon.
